(19) United States
(12) Patent Application Publication
(10) Pub. No.: US 2014/0330087 A1
(43) Pub. Date: Nov. 6, 2014

(54) DEVICES AND METHODS FOR OBTAINING PHYSIOLOGICAL DATA

(71) Applicant: MEDSENSATION, INC., Cambridge, MA (US)

(72) Inventors: Marc Succi, Cambridge, MA (US); Andrew Bishara, Cambridge, MA (US)

(73) Assignee: MEDSENSATION, INC., Cambridge, MA (US)

(21) Appl. No.: 13/968,820

(22) Filed: Aug. 16, 2013

Related U.S. Application Data
(60) Provisional application No. 61/818,409, filed on May 1, 2013.

(51) Int. Cl. A61B 5/00; A61B 5/1455; A61B 8/00; A61B 5/021; A61B 8/02; A61B 8/04; A61B 8/08; A61B 5/0205

(52) U.S. Cl. CPC A61B 5/6826; A61B 8/488; A61B 5/14551; A61B 5/0205; A61B 5/021; A61B 8/02; A61B 8/04; A61B 5/742; A61B 8/462; A61B 5/746; A61B 8/56; A61B 5/02416
USPC 600/301; 600/300; 600/587; 600/453; 600/324; 600/485; 600/438; 600/508; 600/364

(57) ABSTRACT

A medical device that can have at least one sensor for obtaining data relating to at least one physiological condition of a patient. Some embodiments of the medical device can include internet communication which can allow the data obtained by the sensor(s) to be transmitted to a computer monitored by either a physician or the patient.

### REFERENCE TO PRIORITY DOCUMENT

This application claims the benefit of priority under 35 U.S.C. §119(e) of U.S. Provisional Patent Application Ser. No. 61/818,409 filed May 1, 2013 under 37 C.F.R. §1.78(a). Priority of the filing date is hereby claimed and the full disclosure of the aforementioned application is incorporated herein by reference.

### FIELD

The subject matter described herein relates to embodiments of medical devices and methods for monitoring physiological conditions.

### BACKGROUND

Medical devices such as blood pressure monitors are used to monitor conditions such as high and low blood pressure. In some cases, these devices have been adapted for home use and can be used by patients, including non-medical professionals.

### SUMMARY

Disclosed herein are devices and methods related to embodiments of a medical device for monitoring physiological conditions. An embodiment of the medical device may include a body configured to secure to a hand of a user and having at least one finger covering. In addition, the medical device may include at least one sensor positioned on the body, including one or more sensors positioned at a distal end of the at least one finger covering for obtaining data characterizing one or more physiological conditions of a patient.

An embodiment of a method can include using a medical device for obtaining data characterizing one or more physiological conditions of a patient, including securing a medical device to a hand of a user. The medical device can comprise a body configured to secure to a hand of a user and have at least one finger covering and at least one sensor positioned on the body and including one or more sensors positioned at a distal end of the at least one finger covering for obtaining data characterizing one or more physiological conditions of a patient. In addition, the method can include placing at least one sensor against a part of the body of the patient and collecting data associated with the one or more physiological conditions. Additionally, the method can include processing at least a portion of the data and displaying information associated with the data on a display.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE FIGURES

These and other aspects will now be described in detail with reference to the following drawings.

FIG. 1A shows a top view of an embodiment of a medical device applied on a user's hand.

FIG. 1B shows a bottom perspective view of the medical device of FIG. 1A.

FIG. 2 shows the medical device of FIG. 1A being used by a physician for obtaining physiological data from a patient.

FIG. 3 shows the medical device of FIG. 1A being used by a patient at home for obtaining physiological data from the patient.

FIG. 4 shows various features of some embodiments of the medical device, including a display of a computer and a mobile device allowing, for example, a physician to remotely monitor the patient's physiological data obtained by the medical device.

FIG. 5 shows an embodiment of a display showing data obtained by the sensors of the medical device.

FIG. 6 shows another embodiment of a display showing data obtained by the sensors of the medical device.

FIG. 7 shows an embodiment of a medical device having a display showing the patient's sensed blood pressure, blood flow, pulse oxygenation and heart rate.

FIG. 8 shows an embodiment of a Doppler transmitter circuit.

FIG. 9 shows an embodiment of a Doppler receiver circuit.

FIG. 10 shows an embodiment of a pulse oximetry transmitter circuit.

FIG. 11 shows an embodiment of a pulse oximetry receiver circuit.

Like reference symbols in the various drawings indicate like elements.

### DETAILED DESCRIPTION

This disclosure describes embodiments of a medical device for obtaining data associated with at least one physiological condition of a patient and allowing either a physician or another user to monitor the at least one physiological condition. At least some of the physiological conditions, which can be sensed and monitored with the present medical device include blood pressure, blood oxygenation, heart rate and blood flow. In addition, the medical device may be used at the patient's home. Data sensed and collected by the medical device can also be directly or wirelessly uploaded onto one or more remote or local devices, such as a computer, including a cell phone or tablet, which can display the data and allow either the patient or physician to analyze the data, including in real-time, and determine various physiological conditions related to the patient.

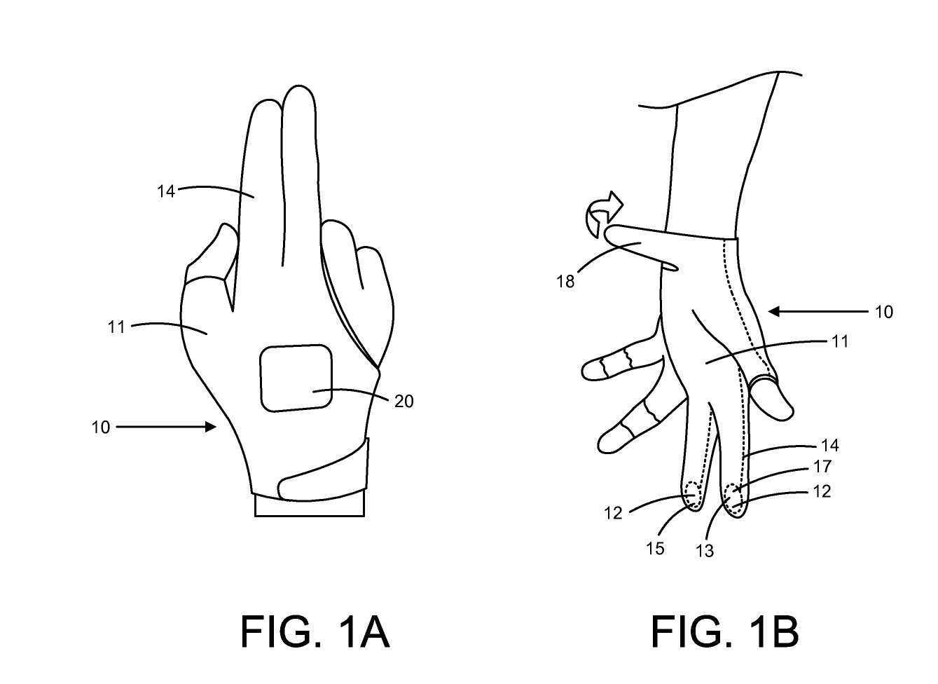

FIGS. 1A and 1B illustrate an embodiment of a medical device 10, which can assist in monitoring the health of a patient. The medical device 10 can include a hand covering or body 11, which can be made out of a variety of materials, including a polyester material. In addition, the medical device 10 can include at least one sensor 12 and circuitry, which can be coupled to or embedded within the material comprising at least a part of the medical device 10. The medical device 10, including the sensors 12 and circuitry, can have a slim configuration, which can allow medical gloves, such as traditional latex gloves, to be worn over the medical device 10.

The body 11 of the medical device 10 can be configured similar to a glove such that the body 11 can secure to a hand of a user and includes at least one finger covering 14, as shown in FIGS. 1A and 1B. Although the embodiment of the medical device 10 is shown in FIGS. 1A and 1B as having two finger coverings 14 that cover the index and middle finger, any number of finger coverings 14 that can cover any number of fingers or portions of fingers of the user can be included in the medical device 10. As shown in FIGS. 1A and 1B, the body 11 can include one or more full or partial finger coverings, such as is shown over the thumb.

Any one of the finger coverings 14 can include at least one sensor 12 secured to the finger coverings 14. For example, the medical device 10 can include one or more sensors 12 on the distal ends of the finger coverings 14, such as over at least one of the index and middle fingers, as shown in FIG. 1B. The position of the sensor 12 at the distal ends of the finger coverings 14 can allow the user to easily apply the sensors 12 against one or more physical features of a patient in order to sense one or more physiological conditions. In addition, one or more sensors 12 can be positioned at various other positions along the body 11 of the medical device 10, including an area of the body 11 configured to cover the palm of the user's hand.

The medical device 10 can sense one or more physiological conditions, such as heart rate, blood pressure, blood oxygenation and blood flow. The medical device 10 can be used, for example, in a hospital or doctor's office as well as at the patient's home in order to obtain physiological data relating to the patient's health.

The one or more sensors 12 of the medical device 10 can assist the user in acquiring a variety of data relating to the health or physiological conditions of the patient. For example, some embodiments of the medical device 10 can include at least one sensor 12 that is force sensor array 13. The force sensor array 13 can detect and obtain data, which can assist in determining at least one of blood pressure, blood flow and heart rate characteristics of the patient.

In addition, some embodiments of the medical device 10 can include at least one Doppler ultrasound transducer 15 and receiver, as shown in FIG. 1B. For example, the Doppler ultrasound transducer 15 and receiver can assist in sensing blood flow within at least one blood vessel of the patient. The Doppler ultrasound transducer 15 can include a transducer having a frequency of approximately 2 MHz to approximately 18 MHz, which can allow the Doppler ultrasound transducer 15 to obtain blood flow waveforms from superficial or deep vessels. For example, higher frequencies can be used in order to obtain blood flow data from more superficial vessels. In addition, blood flow characteristics can be identified using the medical device 10 from a variety of vessels, including superficial vessels, such as the carotid, brachial radial, ulnar, dorsalis pedis, or femoral arteries. Determining blood flow with the Doppler ultrasound transducer 15 can assist in, for example, assessing arterial occlusion, as in patients with peripheral vascular disease.

Some embodiments of the medical device 10 can utilize more than one sensor 12 in order to determine various physiological conditions. For example, the medical device 10 can use sensed data obtained by the Doppler ultrasound transducer 15 and the force sensor array 13 in order to determine blood pressure in a patient.

The Doppler ultrasound transducer 15 can also be used to determine heart rate. For example, the data collected by the Doppler ultrasound transducer 15 can be viewed as a waveform with high amplitude pulses occurring during beats of the heart. The frequency of these pulses can indicate the heart rate of the patient. Therefore, the Doppler ultrasound transducer 15 can assist the medical device 10 with determining at least blood flow, heart rate and blood pressure characteristics of a patient.

In some embodiments of the medical device 10, the sensors 12 can be positioned such that the Doppler ultrasound transducer 15 is located on the middle finger, or finger covering 14, as shown in FIG. 1B, and the force sensor array 13 is located on the index finger, or finger covering 14, as also shown in FIG. 1B. However, any of the sensors 12 can be positioned along any one or more finger coverings 14. In addition, in some embodiments, more than one type of sensor 12 can be positioned along the same finger covering 14.

Some embodiments of the medical device 10 can include at least one pulse oximetry sensor 17 for determining or monitoring the patient's blood oxygen saturation. For example, the pulse oximetry sensor 17 can monitor and obtain data associated with the patient's blood oxygen saturation, or blood oxygenation, by way of either reflectance pulse oximetry or transmissive pulse oximetry. The pulse oximetry sensor 17 can be positioned on any one or more finger coverings 14 of the medical device 10. Additionally, the pulse oximetry sensor 17 can be positioned adjacent to a finger covering 14 having either the force sensor array 13 or Doppler ultrasound transducer 15.

The user can apply any one of the pulse oximetry sensors 17 against a patient, including the user, in order to determine the blood oxygen saturation of at least one underlying blood vessel. In addition, the user can apply various amounts of force with the pulse oximetry sensor 17 against the patient, including a small force, in order to obtain blood oxygen saturation levels.

The blood oxygenation level can vary depending on the state of health of the patient. For example, in congestive heart failure, pulse oxygenation levels may be low due to inadequate blood oxygenation at the level of the lung. Typical pulse oxygenation in health patients can range from approximately 95% to approximately 100%, but can vary significantly between patients.

The one or more sensors 12 of the medical device, including the force sensor array 13, Doppler ultrasound transducer 15 and pulse oximetry sensor 17, can sense data, which can then be collected by a processor or processing system, including a microprocessor, either associated with or integrated with the medical device 10. In addition, data sensed and collected by the medical device 10 can also be directly or wirelessly uploaded onto one or more remote or local devices, such as a computer, including a cell phone or tablet, which can display the data and allow either the patient or physician to analyze the data, including in real-time, and determine various physiological conditions related to the patient. Additionally, the processor or processing system can use the data sensed by the sensors 12 to determine one or more physiological characteristics, such as blood pressure, blood flow, pulse oxygenation, and heart rate.

The medical device 10 can be configured to sense any number of physiological conditions. This can benefit the user by relieving the need to prepare and use more than one device in order to obtain data relating to more than one physiological condition. For example, instead of requiring the use of a blood pressure cuff to obtain blood pressure data and a separate Doppler flow ultrasound device for obtaining blood flow data, the medical device 10 of the present disclosure can obtain at least the blood pressure and blood flow data of the patient. Furthermore, the medical device 10 can obtain and process data relating to other physiological conditions, including at least heart rate and blood oxygen saturation.

In some embodiments, the user, which can include the physician, patient or other individual, can secure the medical device 10 to either the left or right hand. Some embodiments of the medical device can be configured for the left or right hand while some embodiments can be used on either the left or right hand of the user. Once the medical device 10 has been coupled to the hand, the user can wear the medical device 10 prior to, during and after acquiring physiological data from the patient.

The body 11 of the medical device 10 can be applied to the user's hand in a number of ways, including having elastically expandable material that allow the user's hand to enter and fit into the medical device 10. Alternatively or in addition, the body 11 can include one or more straps 18, as shown in FIG. 1B, such as Velcro straps, which can allow the user's hand to enter the body 11 of the medical device 10 as well as secure the body 11 to the user's hand. However, any number of securing mechanisms and body 11 fitting features can be implemented in the medical device 10 for allowing the body 11 to fit securely and comfortably on the user's hand.

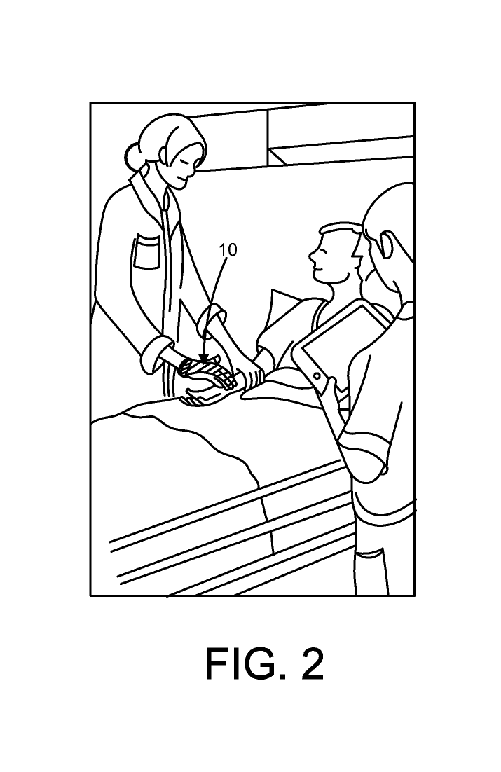

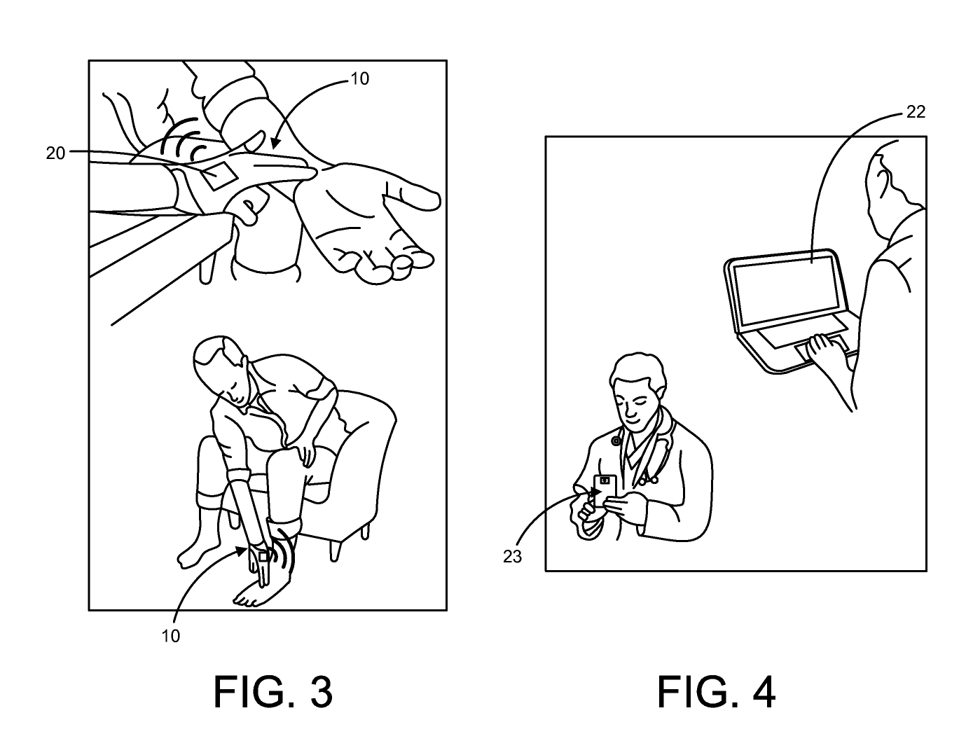

As shown in FIGS. 2 and 3, once the body 11 of the medical device 10 has been applied or secured to the user's hand, the user can then place the one or more sensors 12 against one or more physical features of the patient in order to obtain physiological data. For example, the user can apply at least one sensor 12 against a part of the patient's wrist in order to obtain data associated with at least one of the patient's heart rate, blood pressure, blood flow and blood oxygen saturation associated with one or more vascular structures in the wrist.

Additionally, the user can apply the at least one sensor 12 against the patient's neck in order to obtain data associated with at least one of the patient's heart rate, blood pressure, blood flow and blood oxygen saturation associated with one or more vascular structures in the neck. The sensors 12 can be placed against any number of parts of the body in order to obtain a variety of physiological data.

The configuration of the medical device 10, including the positioning of the sensors 12, such as at the distal end of the finger coverings 14, can allow the medical device 10 to be versatile in its use. For example, once the medical device 10 has been coupled to the user's hand, the user can then extend one or more fingers and apply the one or more sensors 12 positioned at the distal end of the one or more finger coverings 14 against the body of the patient in order to obtain physiological data. In addition, the user can use the medical device 10 to obtain data at more than one position along the body, including the wrist, neck, foot, lower leg and chest, without having to use more than one medical device 10. In contrast, other medical devices such as blood pressure cuffs can require either alterations or alternate devices in order to accommodate different sized patients and are restricted to obtaining blood pressure data to select parts of the body, such as at the upper arm and ankle.

The medical device 10 can be configured to obtain blood pressure data from any blood vessel and is not limited to obtaining blood pressure to only the upper arm or lower leg. For example, the user can extend one or more fingers and apply the sensors 12 against any part of the patient's body in order to obtain blood pressure data associated with the vasculature in that part of the body. Furthermore, any number of additional physiological data, such as blood flow, heart rate and blood oxygen saturation, can be obtained from any part of the body of interest. Therefore, the medical device 10 allows a user to obtain a variety of physiological data from a variety of parts of the patient's body. Additionally, the medical device can be used to obtain physiological data across a variety of sized and shaped patients of either sex and of any age.

Some embodiments of the medical device 10 can include a processor or processing system, including a microprocessor, which can perform at least one of retrieving, storing and analyzing the data obtained by the sensors 12. The processor or processing system can provide several advantages, including the ability to easily store data obtained by the sensors 12 as well as provide the ability to easily compare and analyze the data obtained by the sensors 12. The ability to store, compare and analyze data obtained by the sensors 12 can allow either the physician or patient to effectively monitor the patient's health. In addition, compiling and analyzing the data obtained by the medical device 10 can assist in allowing large scale analytics of the patient's health as well as discovering physiological changes of the patient.

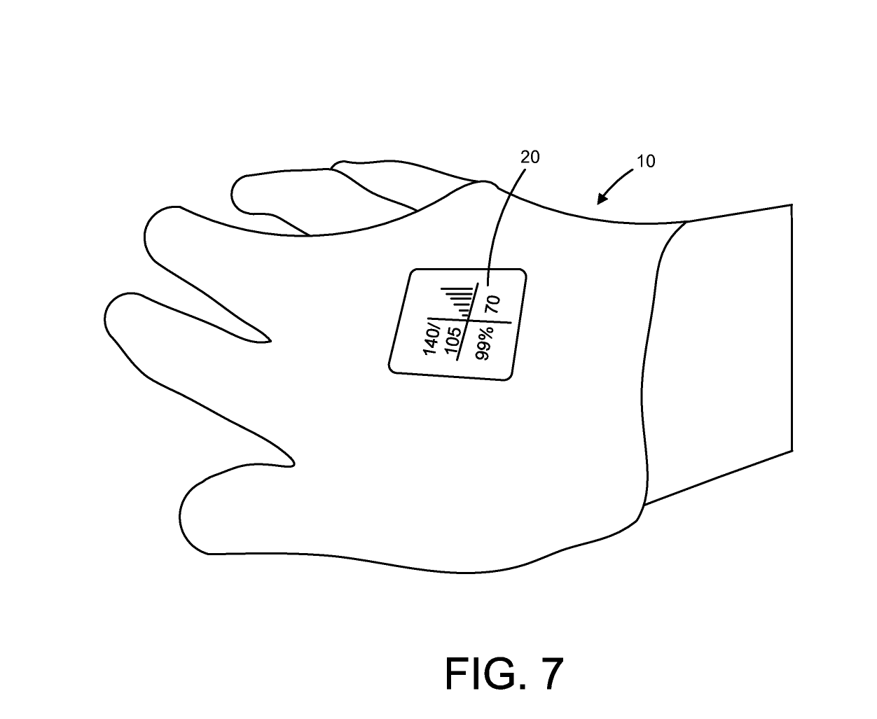

As shown in FIGS. 3 and 7, some embodiments of the medical device 10 can include at least one display feature 20 for assisting in providing patient related information, including one or more real-time physiological conditions of the patient. The display feature 20 can provide either detailed information about the data obtained from the sensors 12 or a general overall patient status. The display feature 20 can include a small LCD screen secured to, including incorporated into, a side of the body 11.

For example, as shown in FIGS. 3 and 7, the display feature 20 can be positioned on the body 11 such that it is located on the top side of the user's hand during use. This positioning can allow the display feature 20 to be viewed during use of the medical device 10, as shown, for example, in FIG. 3. The display feature 20 can provide information without the use of a data connection and the information displayed on the display feature 20 can optionally be transmitted, either wirelessly or directly, to one or more data platforms, including the processing system discussed above.

Some embodiments of the medical device 10 can include either direct or wireless internet communication features. In addition, the internet communication features can allow the medical device 10 to transmit data to a processing and/or display interface, such as a computer 22, including a mobile computing device such as a cell phone 23, as shown in FIG. 4, which can provide real-time assessment of the health of the patient. Computer 22 may be a laptop as shown, or could be other types of computing devices such as servers, tablets or cell phones, and may be connected through Wi-Fi, Bluetooth, the internet, etc.

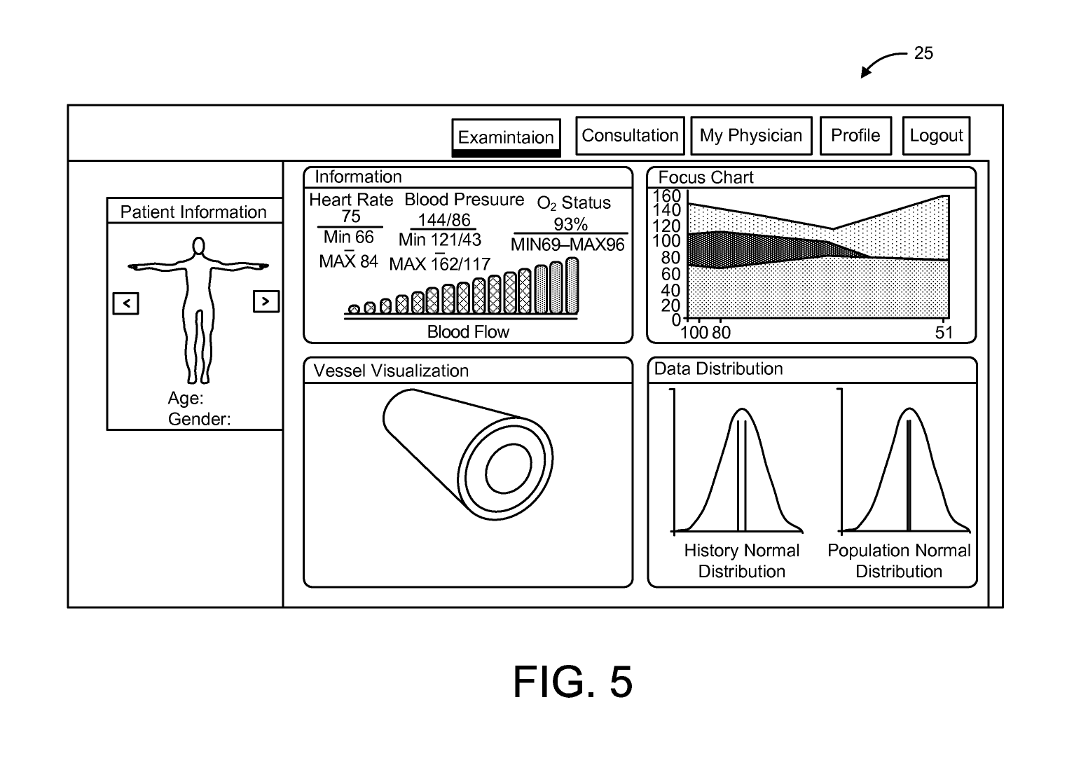

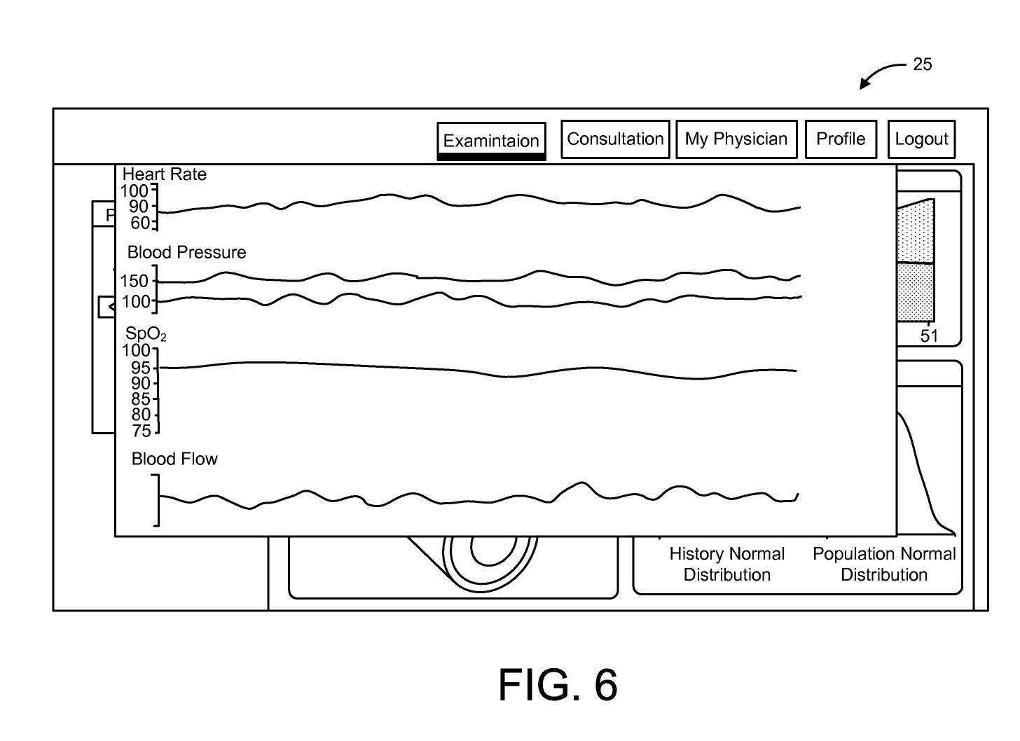

FIGS. 5 and 6 show example embodiments of a display 25 provided by computer 22. The display 25 can provide a variety of information collected and computed by the medical device 10, which can then be analyzed and monitored by either the physician or the patient. In addition, the information provided on display 25 can provide information related to the patient in at least near real-time. Physiological data relating to one or more physiological conditions of the patient may also be monitored by the physician remotely.

The computer 22 can present the data sensed and collected by the one or more sensors 12 in a variety of ways, which the physician can use to analyze and monitor the health of the patient. For example, as shown in FIG. 5, the display 25 of computer 22 can include one or more pieces of patient related data such as vascular conditions, data history, heart rate, blood pressure, and blood oxygenation.

In some embodiments, an estimate of vessel lumen size or condition, as shown in the vessel visualization of FIG. 5, can be estimated by combing data obtained from one or more sensors 12, such as blood flow data obtained by the Doppler ultrasound transducer 15 and force data obtained by one or more force sensor arrays 13, as well as data calculated by the processor, such as calculated data characterizing the elasticity of the blood vessel using Young's modulus.

The display 25 can also include one or more charts or graphs in order to show, for example, data collected over time, as shown in FIG. 6. In addition, display 25 can include a variety of patient information such as medical history, gender, age and name of the patient. Any number of types of data displayed in one or more of a variety of ways can be conveyed on the display 25 for monitoring and analyzing one or more physiological conditions of the patient.

In addition, the computer 22 can include an alert system, which can alert either the physician or patient of any abnormal data or data points obtained by the medical device 10. Additionally, the display feature 20 or an audible alarm can provide an alert to the user of the medical device 10 that abnormal data has been obtained, such as high blood pressure or absent blood flow. This can provide either the patient or physician with important health information relating to the patient, which can allow improved response and medical care to the patient.

Additionally, in some embodiments, the processing system can include a reinforcement feature that can improve data acquisition and internal data models of vessel compliance, which can be updated during data collection in order to optimize data acquisition from the one or more sensors 12. For example, if a user applies a force against a blood vessel, such as with one or more sensors 12, which is overly constrictive of the blood vessel, the medical device 10 can provide an alert to the user. The alert can at least inform the user to reduce the amount of applied force in order to facilitate improved data collection from the sensors 12 sensing one or more characteristics of the blood vessel. In addition, if the force applied is inadequate to achieve proper data acquisition, the medical device 10 may provide an alert that can inform the user to increase the applied force in order to facilitate improved data collection.

For example, in order to collect certain physiological data, a greater amount of force applied to the blood vessel may be required, such as in order to cause occlusion of the blood vessel. In such as case, the medical device 10 can provide an alert to the user if occlusion is not established. In addition, if the user applies an inconsistent force, the medical device 10 can alert the user to improve the consistency of applied force.

In some embodiments, data obtained by the Doppler ultrasound transducer 15 can be analyzed by the medical device 10, such as by the processor, in order to ensure that blood flow can be sufficiently obtained prior to applying force with at least one sensor 12. In addition, an alert provide information to a user regarding the need to reposition the one or more sensors 12 in order to have the one or more sensors 12 properly positioned for obtaining data, such as over a blood vessel having sufficient blood flow.

Some embodiments of the medical device can also be configured to obtain one or more of a variety of data or data points in approximately less than 10 seconds. In addition, some embodiments of the medical device 10 can include an analytics system, which is capable of displaying trending data over time and can be adjustable by one or more time periods.

A physician can be alerted remotely by computer 22 regarding a physiological condition, such as high blood pressure and can direct the patient to alter medication type or dose in order to improve the physiological condition or call emergency services. Therefore, the physician can both monitor and care for patients remotely with the use of the medical device 10.

FIG. 7 shows an embodiment of the medical device 10 with a display feature 20, such as a liquid crystal display (LCD) screen, displaying sensed data from a variety of sensors 12. In addition, the sensed data can be displayed on the display feature 20 such that the data can be easily interpreted and understood by a user viewing the display feature 20. For example, the display feature 20 can simultaneously show data associated with blood pressure, blood flow, pulse oxygenation and heart rate of the patient, as shown in FIG. 7, including one or more in at least near real-time. In addition, the data shown on the display feature 20 can also be shown on the computer 22, which can also display the data sensed by the sensors 12 in at least near real-time.

The medical device 10 can include one or more of a variety of sensors 12, processors, transmitters and circuitry in order to allow the medical device 10 to include a variety of functions for collecting, analyzing and displaying one or more physiological conditions associated with at least one patient. In addition, the medical device 10 can have a compact configuration for allowing ease of use.

In some embodiments, the medical device 10 can include circuitry, which can be integrated within the body 11, such as within the material that makes up at least a part of the body 11. This can allow wires and electrical components to be protected and confined within the body 11 of the medical device 10.

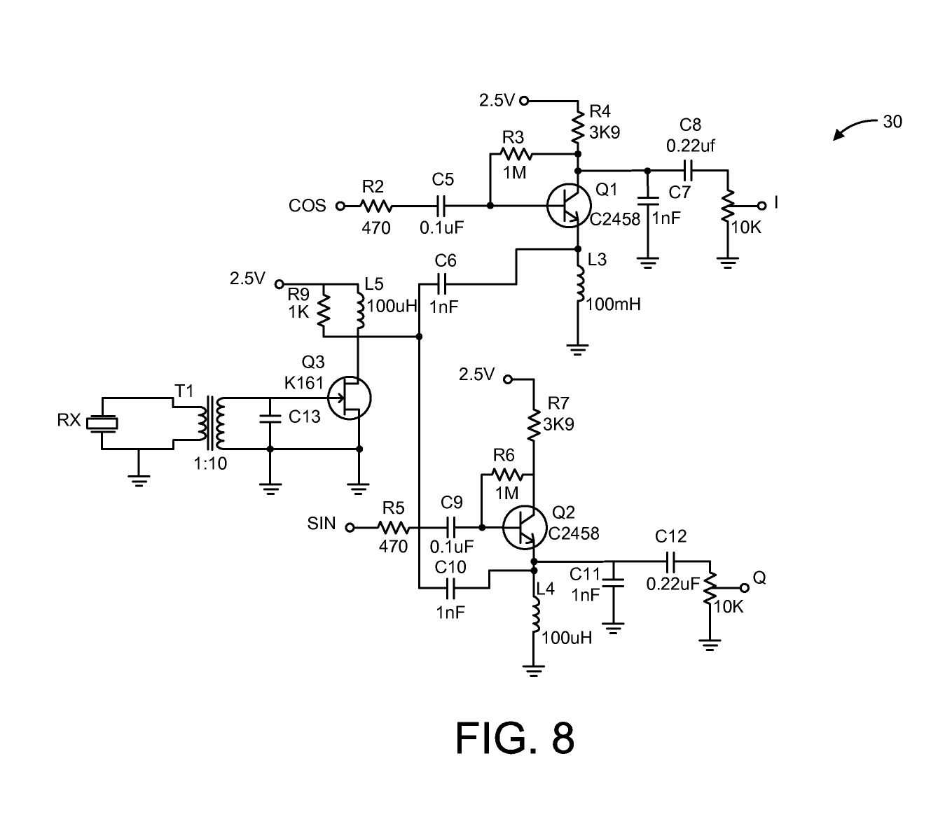

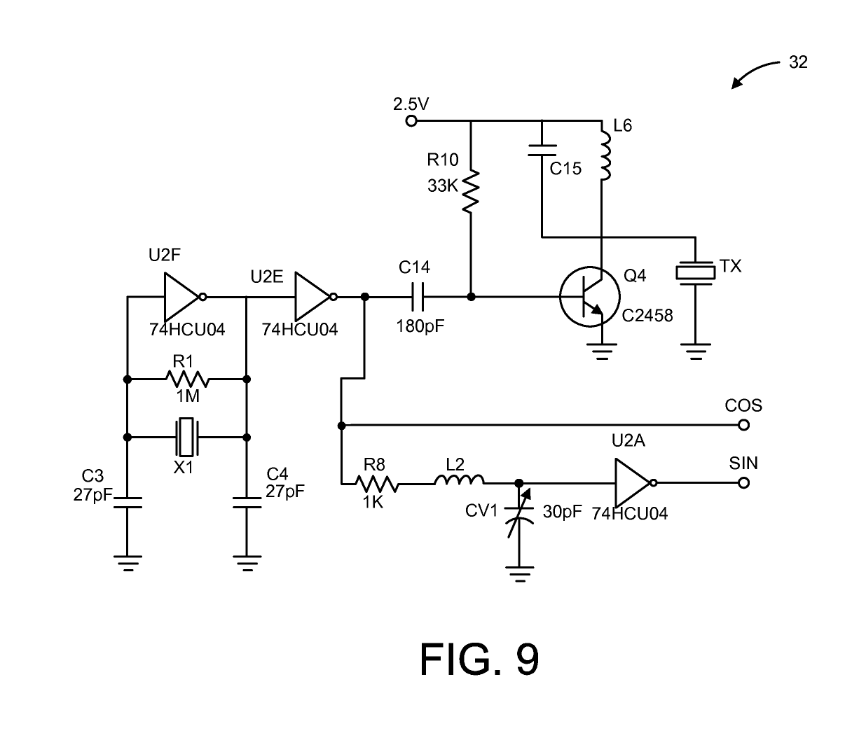

FIG. 8 shows an embodiment of a Doppler ultrasound transmitter circuit 30 and FIG. 9 shows an embodiment of a Doppler ultrasound receiver circuit 32. These circuits can be integrated in the body 11 of the medical device 10 and can assist in allowing the Doppler ultrasound sensor 15 to sense and collect data, which can then be displayed on either the display feature 20 associated with the body 11 of the medical device or the display 25 of the computer 22. In addition, the circuitry shown in FIGS. 8 and 9 can be implemented in a variety of ways in order to allow the Doppler ultrasound sensor 15 to sense one or more physiological characteristics of the patient.

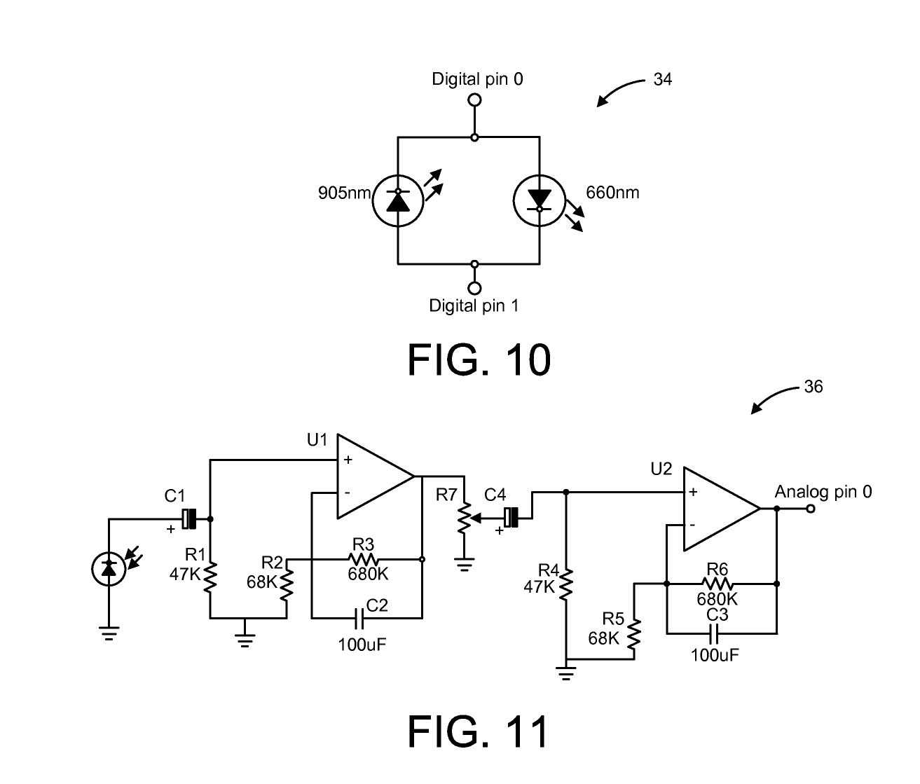

FIG. 10 shows an embodiment of a pulse oximetry transmitter circuit 34 and FIG. 11 shows an embodiment of a pulse oximetry receiver circuit 36. These circuits can also be integrated in the body 11 of the medical device 10 and can assist in allowing the pulse oximetry sensor 17 to sense and collect data, which can then be displayed on either the display feature 20 associated with the body 11 of the medical device or the display 25 of the computer 22. In addition, the circuitry shown in FIGS. 10 and 11 can be implemented in a variety of ways in order to allow the pulse oximetry sensor 17 to sense one or more physiological characteristics of the patient.

Various mathematical and physics concepts can be incorporated into the medical device 10 in order to determine a variety of physiological conditions of the patient, including blood pressure, blood flow, blood oxygen saturation and heart rate. For example, the medical device 10 can determine the blood pressure in a patient by estimating the amount of stress and change in radius of the blood vessel that sensor 12 is placed adjacent to and obtains data from.

Some methods of use of the medical device 10 include the user placing at least one sensor 12 of the medical device 10 against the patient's body, such as against the skin of the patient. Sensor 12 may be positioned against the patient's body such that the at least one sensor 12 is adjacent a blood vessel. Additionally, the user can apply one or more of a variety of pressures against the blood vessel with the sensor 12 in order to obtain data or data points corresponding to one or more physiological conditions, such as heart rate, blood flow, blood pressure and blood oxygenation.

The medical device 10 can process the sensed data with the processor or processing system in order to provide information to either the user or patient regarding one or more physiological conditions, such as heart rate, blood flow, blood pressure and blood oxygenation. In addition, this information can be displayed on the display 25, as discussed above.

Alternatively or in addition, the sensed data can be processed by the processor in order to estimate the amount of stress and change in radius of the blood vessel, such as an artery, by pushing on the blood vessel wall with the sensor 12. This can be accomplished by pushing the sensor 12 against the skin of the patient directly over or adjacent to the blood vessel. The application of pressure against the vessel can provide information relating to approximately 90 degrees of the artery and approximately 0.5 cm to approximately 1.5 cm in length. The medical device 10 can provide an estimate amount of stress and change in radius over an area, which can be calculated using an algorithm incorporated in the medical device 10 that calculates area, such as equation 1.

Area=¼(2πr)(length) Equation 1

In addition, the medical device 10 can consider the elasticity of the blood vessel when estimating blood pressure. Additionally, the medical device 10 can determine the blood pressure in the vessel by taking into account the amount of pressure being applied by the sensor 12 against the vessel. For example, the medical device 10 can include an algorithm for factoring in the amount of pressure applied to the vessel when determining the blood pressure in the vessel, such as equations 2 and 3.

pressure inside vessel=(blood pressure)−(pressure applied by hand) Equation 2

pressure applied by hand=f/(¼(2πr)(length) Equation 3

In some embodiments, the stress in the vessel can change from σ=pD/2t into σ=(p−f/(¼(2πr)(length)))D/2t. This can be for ¼part of the vessel, such as where a force or pressure is applied, and where the changes can make the stress slightly decrease. In general, the decrease in stress in the vessel can result in a decrease in radius, which can result in a change in resistance of the vessel. Therefore, the medical device 10 can measure a change in flow and determine the resistance of the vessel.

The steps above for determining blood pressure or resistance of the vessel can be repeated one or more times in order to confirm the results. In addition, the above steps can be repeated under various conditions, including different amounts of applied pressure against the vessel. If the results obtained are consistent, then the medical device can at least one of store, display or analyze the blood pressure readings. If the results obtained are not consistent, then at least one algorithm or model can be modified until consistent results are obtained.

Additionally, the medical device 10 can determine the blood pressure in a vessel during systole and diastole. For example, the user can apply a moderate amount of force against the vessel, such as with the force sensor array 13, until blood flow generally stops. The Doppler ultrasound transducer 15 can then be laid over (without applying substantial force) the same vessel as the force sensor array 13, but slightly more distal relative to the direction of blood flow, and record blood flow waves utilizing continuous or pulse wave Doppler ultrasound. In conjunction with pressure information from the force sensor array 13, recording the changes from the Doppler wave form can indicate both systolic and diastolic blood pressure.

For example, when complete occlusion of the blood vessels is achieved by the force sensor array 13, the force at occlusion can be recorded. If Doppler ultrasound transducer 15 overlies the same blood vessel in a slightly more distal position, it can record the absence of the waveform. As the finger applying the force via sensor is varied in force applied, the Doppler ultrasound transducer 15 can record at what point the blood flow waveform completely stops, and associate this point with the force applied by the force sensor array 13. This may be referred to as the systolic blood pressure.

After systolic blood pressure is obtained, the force applied by the force sensor array 13 can be gradually reduced by the user as the user lifts the finger associated with the force sensor array 13 off the blood vessel. For example, at approximately the same time that the user lifts the finger associated with the force sensor array 13, the Doppler ultrasound transducer 15 overlying the same blood vessel can record the return of normal vessel waveforms, which can indicate the diastolic blood pressure. The data can then be compared to previous measurements of blood pressure in order to determine if the data is consistent.

Although a few specific embodiments have been described in detail above, other modifications consistent with the spirit of this disclosure are contemplated.

1. A medical device comprising:
a body configured to secure to a hand of a user and having at least one finger covering;
at least one sensor positioned on the body, including one or more sensors positioned at a distal end of the at least one finger covering for obtaining data characterizing one or more physiological conditions of a patient.

2. The medical device of claim 1, wherein the at least one sensor includes one or more of a force sensor array, Doppler ultrasound transducer, and pulse oximetry sensor.

3. The medical device of claim 2, further including a processor configured to determine one or more of a blood pressure, blood flow, heart rate, and blood oxygen saturation using data obtained from the at least one sensor.

4. The medical device of claim 2, further comprising: a processor configured to perform at least one of retrieving, storing and analyzing data obtained by the at least one sensor and converting the data into information characterizing one or more physiological conditions of the patient; and
a display configured to convey the information to a user.

5. The medical device of claim 4, wherein the information on the display is displayed in at least near-real time.

6. The medical device of claim 1, further including a display secured to the body.

7. The medical device of claim 6, wherein the display conveys information relating to one or more physiological conditions of the patient to a user.

8. The medical device of claim 7, wherein the information is displayed in at least near real-time.

9. The medical device of claim 1, further including an alarm system for alerting a user when abnormal data has been obtained by at least one sensor.

10. The medical device of claim 1, wherein data obtained by the at least one sensor is directly or wirelessly transmitted to a computer.

11. The medical device of claim 10, wherein the computer includes a display which conveys patient related information, including one or more of a vascular condition, data history, heart rate, blood pressure, and blood oxygenation.

12. The medical device of claim 1, wherein the processor includes an algorithm that utilizes the amount of pressure applied to a blood vessel with the at least one sensor to determine blood pressure in the vessel.

13. A method of using a medical device for obtaining data characterizing one or more physiological conditions of a patient comprising:
securing a medical device to a hand of a user, wherein the medical device comprises a body configured to secure to a hand of a user and having at least one finger covering and at least one sensor positioned on the body, including one or more sensors positioned at a distal end of the at least one finger covering for obtaining data characterizing one or more physiological conditions of a patient;
placing at least one sensor against a part of the body of the patient;
collecting data associated with the one or more physiological conditions;
processing at least a portion of the data; and
displaying information associated with the data on a display.

14. The method of claim 13, wherein the at least one sensor includes one or more of a force sensor array, Doppler ultrasound transducer, and pulse oximetry sensor.

15. The method of claim 14, wherein the medical device further includes a processor configured to determine one or more of a blood pressure, blood flow, heart rate, and blood oxygen saturation using data obtained from the at least one sensor.

16. The method of claim 14, wherein the medical device further comprises: a processor configured to perform at least one of retrieving, storing and analyzing data obtained by the at least one sensor and converting the data into information characterizing one or more physiological conditions of the patient; and
a display configured to convey the information to a user.

17. The method of claim 16, wherein the information on the display is displayed in at least near-real time.

18. The method of claim 13, wherein the medical device further includes a display secured to the body.

19. The method of claim 18, wherein the display conveys information relating to one or more physiological conditions in at least near real-time.

20. The method of claim 13, wherein the medical device further includes an alarm system for alerting a user when abnormal data has been obtained by at least one sensor.

21. The method of claim 13, wherein data obtained by the at least one sensor is directly or wirelessly transmitted to a computer.

22. The method of claim 21, wherein either the computer includes a display which conveys patient related information, including one or more of a vascular condition, data history, heart rate, blood pressure, and blood oxygenation.

23. The method of claim 13, further including processing an algorithm that utilizes the amount of pressure applied to a blood vessel with the at least one sensor to determine blood pressure in the vessel.

24. The method of claim 13, further including applying a force sensor array and Doppler ultrasound transducer against a blood vessel and recording changes in data received from the Doppler ultrasound transducer indicating systolic blood pressure and diastolic blood pressure.
